(19) United States
(12) Patent Application Publication
(10) Pub. No.: US 2021/0069089 A1
(43) Pub. Date: Mar. 11, 2021

(54) STYLING COMPOSITION FOR TEMPORARILY DEFORMING KERATINOUS FIBERS AND ITS USE

(71) Applicant: Henkel AG & Co. KGaA, Duesseldorf (DE)

(72) Inventors: THORSTEN KNAPPE, Schenefeld (DE); MARGAUX LEFEBVRE, Charney-lés-Macon (FR)

(73) Assignee: Henkel AG & Co. KGaA, Duesseldorf (DE)

(21) Appl. No.: 17/007,859

(22) Filed: Aug. 31, 2020

(30) Foreign Application Priority Data
Sep. 6, 2019 (DE) 10 2019 213 571.4

(51) Int. Cl. A61K 8/73; A61K 8/9789; A61Q 5/06

(52) U.S. Cl. CPC A61K 8/732; A61K 8/737; A61Q 5/06; A61K 8/73; A61K 8/9789

(57) ABSTRACT

The notification concerns a styling composition for temporary shaping of keratinous fibers comprising

a) at least one modified starch, preferably a dextrin,

b) at least one natural plant exudate and/or extract, and

c) at least one guar derivative.

### CROSS-REFERENCE TO RELATED APPLICATION

This application claims priority to German Patent Application No. 10 2019 213 571.4, filed Sep. 6, 2019, which is incorporated herein by reference in its entirety.

### TECHNICAL FIELD

The present disclosure comprises a styling composition for temporary shaping of keratinous fibers and its use.

### BACKGROUND

Styling compositions for the temporary reshaping of keratinous fibers, preferably hair, are known as styling products and are usually used in the form of gels, pomades, wax, foam, and spray.

Keratinic fibers comprise all fibers containing keratin, such as wool, furs and feathers, especially human hair, with emphasis on human hair. During a temporary reshaping of keratinous fibers, no chemical (oxidation and reduction) process takes place. Instead, the fibers are shaped by applying styling products containing firming agents such as waxes and/or polymers.

To fix hairstyles without negatively affecting the shine and healthy, natural look of the hair, common styling products often contain synthetic polymers. These are often produced from synthetic monomers obtained by refining fossil raw materials, such as crude oil, with increased energy consumption.

As part of a more conscious approach to nature and the finite nature of raw material deposits, the demand of end consumers for natural, sustainable products, i.e. products produced using regenerative raw materials that use as little energy as possible, is increasing.

Therefore, the aim of current research is to find natural components which, when used in compositions for the temporary reshaping of keratinous fibers, have the same positive properties as the synthetic polymers used so far. In addition to excellent hold, they should give the hairstyle additional shine, bounce and suppleness and avoid the formation of a visible film on the hair, which makes it look dull. A further requirement for these natural raw materials is their processability into stable and user-friendly compositions.

### BRIEF SUMMARY

It is therefore the task of the present disclosure to provide a composition for the temporary shaping of keratinous fibers, which contains predominantly natural ingredients and offers the same hold, shine, and suppleness as known styling products with synthetic components.

In one embodiment, the disclosure provides a styling composition for temporarily deforming keratinous fibers comprising
a) at least one modified starch,
b) at least one natural plant exudate and/or extract, and
c) at least one guar derivative.

In another embodiment, the disclosure provides styling composition for temporarily deforming keratinous fibers comprising
a dextrin present in an amount of from about 2.0 to 4.0% by weight, based on the total proportion of the styling composition,
acacia exudate and/or extract present in an amount of from about 1.5 to 3.0% by weight, based on the total proportion of the styling composition, and
guar hydroxypropyltrimonium chloride present in an amount of from about 0.2 to 0.5% by weight, based on the total proportion of the styling composition,
at least one protein hydrolysate and/or one protein hydrolysate derivative present in an amount of from about 0.01 to 0.35% by weight, based on the total proportion of the styling composition, and
one or more components chosen from surfactants, preservatives, humectants, pH adjusting agents, care agents, dyes, conditioning agents, perfume, and mixtures thereof.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The task is solved by a styling composition for temporary deformation of keratinous fibers, comprising

a) at least one modified starch, preferably a dextrin,
b) at least one natural plant exudate and/or extract, and
c) at least one guar derivative.

The styling composition is a cosmetic product, which can be present as a usual formulation of styling products and can be packaged, for example as a gel, wax, paste, lotion, mouse or spray, preferably the styling composition is formulated as a hair dryer spray.

Preferably the styling composition is aqueous. Aqueous styling composition means in the sense of the present disclosure a composition whose cosmetically acceptable carrier component is water, preferably distilled water, especially distilled water without hydrogen peroxide (H₂O₂). The proportion of water in the styling composition is preferably from about 55 to about 98% by weight, particularly preferably from about 60 to about 95% by weight, very preferably from about 70 to about 90% by weight, even more preferably from about 80 to about 90% by weight, based on the total proportion of the styling composition.

In a particularly preferred embodiment as contemplated herein, the styling composition comprises sufficient water to complete the remaining components contained in the styling composition to about 100% by weight, measured on the total weight of the styling composition, also referred to as “ad. 100”.

The sum of all components of the styling composition is about 100% by weight, based on the total proportion of the styling composition.

Starch, also known as poly-α-glucose, is a reserve carbohydrate that is stored by many plants in the form of starch grains (granules), usually from about 1 to about 200 μm in size, in various parts of the plant, e.g. in tubers or roots, cereal seeds, fruits as well as in the pith. In plants, starch is essentially present in two different polymers, namely amylopectin and amylose.

Amylose includes predominantly linear α-1,4-glycosidically linked D-glucose. The resulting chains form double helices in thickness. Higher plants contain from 0 to about 45% by weight of amylose based on dry substance.

In addition to the α-1.4-linkages described for amylose, amylopectin also contains α-1.6-bonds as branching sites in an amount of from about 4 to about 6%. The average distance between the branching points is about from about 12 to about 17 glucose units. The molar mass of from about 10⁷ to about 7*10⁸ corresponds to approximately 10⁵ glucose units, making amylopectin one of the largest biopolymers. These branches are distributed over the molecule in such a way that a tuft structure with relatively short side chains develops. Two of these side chains each form a double helix. Due to the many branching points, amylopectin is relatively well soluble in water.

Modified starches can be obtained by chemical, enzymatic or physical processes. The modified starch is preferably obtained by hydrolysis, even more preferably by treatment with lye, such as caustic soda. Particularly preferred starch derivatives are the degradation products of starch, such as dextrins, especially e.g. white dextrins, yellow dextrins or maltodextrins. But other modified starches are also suitable, such as partially degraded starches, swelling starches, soluble starches, or hydrolytically degraded starches. Furthermore, non-ionic, anionic, or cationic starch ethers, e.g. starch carboxymethyl ether, hydroxyethyl starches, hydroxypropyl starches can also be used.

As contemplated herein, maltodextrins, such as those available under the trade name Agenamalt, are particularly preferred. Maltodextrins are a mixture of monomers, dimers, oligomers, and polymers of glucose obtained by preferentially acid or enzymatic cleavage of starch, the exact composition varying according to the degree of hydrolysis of the starches. According to the guideline for starch products of the Federal Food Law and Food Science, products with a dextrose equivalent of from about 3 to about 20 are called maltodextrin, whereby the dextrose equivalent indicates the percentage by mass of glucose.

Preferred starches to be modified or modified starches originate from potatoes, corn, rice, peas, acorns, chestnuts, barley, wheat, bananas, sago, millet, sorghum, oats, barley, rye, beans, potato, maranta, manioc or mixtures of these, potato starch is particularly preferred, tapioca starch is even more preferred.

Preferably at least one natural plant exudate and/or extract is an acacia exudate and/or extract.

Exudation is the excretion of mixtures of substances, for example comprising monosaccharides, amino acids, and other small molecular organic compounds, from the plant. The excretion products obtained, such as resins, waxes, and plant juices, are called exudate or plant exudate.

For the purposes of the present disclosure, “extract” means any mixture of substances obtained from a plant by employing an aqueous or organic, preferably alcoholic, extraction agent.

Preferred is the dried exudate of the acacia, which is called gum arabic. Those species of acacia that occur in the tropical and subtropical regions of Africa, India, Central and North America—such as the species Acacia Senegal, Acacia seyal, Acacia karroo and Acacia laeta—are preferred, the most important being the species Acacia Senegal, which is native to the southern regions of the Nile. In a configuration of the present disclosure, the acacia exudate and/or extract is a mixture of exudates and/or extracts of the above-mentioned species.

Gum arabic is preferably present as a branched polysaccharide whose main chain includes ß(1->3)-branched D-galactopyranose units. The main components as building blocks of gum arabic are L-rhamnose, L-arabinose, D-galactose, and D-glucuronic acid. As contemplated herein, preferably suitable gum arabic has a weight-average molar mass of from about 200,000 to about 1,200,000 g/mol, particularly preferred from about 300,000 to about 700,000 g/mol, particularly preferred from about 330,000 to about 550,000 g/mol.

In a preferred embodiment as contemplated herein, the styling composition contains at least one additional plant extract and/or exudate. Preferably at least one other plant extract and/or plant exudate chosen from birch sap, coconut milk, hop extract, avocado extract, chamomile extract and mixtures of these. Also suitable are the extracts and/or exudates from plants or parts of plants of green tea, oak bark, nettle, witch hazel, burdock root, horsetail, hawthorn, lime blossom, lychee, almond, aloe vera, spruce needle, horse chestnut, sandalwood, juniper, coconut, mango, Apricot, lime, wheat, kiwi, melon, orange, grapefruit, sage, rosemary, mallow, cuckoo flower, thyme, lemon balm, cowslip, marshmallow, ginseng, ginger root, Echinacea purpurea, Olea europea, Boerhavia diffusa roots, Foeniculum vulgaris and Apim graveolens.

Preferably the at least one other plant extract and/or exudate with a proportion of from about 0.5 to about 15.0% by weight, preferably from about 1.0 to about 10.0% by weight, particularly preferably from about 2.0 to about 7.0% by weight, even more preferably from about 5.5 to about 6.5% by weight, based on the total proportion of the styling composition, in the styling composition.

Preferably the at least one guar derivative is a cationic guar derivative, preferably guar hydroxypropyltrimonium chloride.

Guar is a polysaccharide extracted from the seed of the guar bean. Preferably the guar derivative is a cationic guar compound (derivative), especially preferred guar derivatives are the cationic hydroxyalkyl guar derivatives, preferably cationic hydroxyethyltrimethylammonium guar and/or cationic hydroxypropyltrimethylammonium guar with average molecular weights of from about 300,000 to about 2,500,000 Daltons. Especially preferred are the cationic guar polymers known under the INCI designation Guar Hydroxypropyltrimonium Chloride with a molecular weight (weight average) of from about 900,000 to about 3,600,000 Daltons, preferably from about 300,000 to about 2,500,000 Daltons. The cationic charge density of these guar polymers is preferably at least about 0.5 meq/g, preferably at least about 0.6 meq/g and especially at least about 0.8 meq/g. Their nitrogen content is preferably in the range from about 1.1 to about 1.6% by weight (based on their total weight).

Suitable cationic guar compounds are sold under the trade name Jaguar® and have the INCI designation Guar Hydroxypropyltrimonium Chloride. Furthermore, particularly suitable cationic guar compounds are also available from Hercules under the name N-Hance®. Other cationic guar compounds are marketed by BASF SE under the name Cosmedia®. A preferred cationic guar compound is the commercial product AquaCat® from the company Hercules. This raw material is an already pre-dissolved cationic guar compound.

Preferably the at least one modified starch, preferably a dextrin, with a proportion of from about 1.0 to about 8.0% by weight, preferably from about 1.5 to about 6.5% by weight, particularly preferably from about 2.0 to about 5.0% by weight, even more preferably from about 2.0 to about 4.0% by weight, based on the total proportion of the styling composition, in the styling composition.

Preferably the at least one natural plant exudate and/or extract with a proportion of from about 0.1 to about 7.0% by weight, preferably from about 0.5 to about 5.5% by weight, particularly preferably from about 1.0 to about 4.0% by weight, even more preferably from about 1.5 to about 3.0% by weight, based on the total proportion of the styling composition, in the styling composition.

Preferably the at least one guar derivative with a proportion of from about 0.01 to about 1.5% by weight, preferably from about 0.08 to about 1.0% by weight, particularly preferably from about 0.1 to about 0.8% by weight, even more preferably from about 0.2 to about 0.5% by weight, based on the total proportion of the styling composition.

Preferably the styling composition contains at least one protein hydrolysate and/or one protein hydrolysate derivative.

Protein hydrolysates are product mixtures obtained by acidic, basic, or enzymatic catalyzed degradation of proteins. As contemplated herein, the term protein hydrolysates also includes total hydrolysates as well as individual amino acids and their derivatives and mixtures of different amino acids. The molecular weight of protein hydrolysates which can be used as contemplated herein is between about 75 daltons, the molecular weight for glycine, and about 200,000 daltons, preferably the molecular weight is from about 75 to about 50,000 daltons and most preferably from about 75 to about 20,000 daltons.

Furthermore, the protein hydrolysates used may be of plant, animal, and/or marine origin. Animal protein hydrolysates include elastin, collagen, keratin, silk, and milk protein hydrolysates, which may also be in the form of salts. Such products are sold under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda). Protein hydrolysates of vegetable origin are preferred, e.g. soya, almond, rice, pea, potato, and wheat protein hydrolysates, preferably wheat protein, almond protein, and mixtures thereof. Such products are available under the trademarks Gluadin® (Cognis), DiaMin® (Diamalt), Lexein® (Inolex) and Crotein® (Croda).

Cationised protein hydrolysates can also be used, whereby the underlying protein hydrolysate can be derived from animals, for example from collagen, milk or keratin, from plants, for example from wheat, maize, rice, potatoes, soya or almonds, from marine life forms, for example from fish collagen or algae, or from biotechnologically produced protein hydrolysates. The protein hydrolysates underlying the cationic derivatives can be obtained from the corresponding proteins by chemical hydrolysis, alkaline or acid hydrolysis, by enzymatic hydrolysis and/or a combination of both types of hydrolysis. The hydrolysis of proteins usually results in a protein hydrolysate with a molecular weight distribution of about 100 Daltons up to several thousand Daltons. Preference is given to those cationic protein hydrolysates whose underlying protein fraction has a molecular weight of from about 100 up to about 25000 Daltons, preferably from about 250 to about 5000 Daltons. Furthermore, cationic protein hydrolysates are quaternized amino acids and mixtures thereof. Quaternization of the protein hydrolysates or amino acids is often carried out using quaternary ammonium salts such as N,N-dimethyl-N-(n-alkyl)-N-(2-hydroxy-3-chloro-n-propyl)-ammonium halides. Furthermore, the cationic protein hydrolysates can also be further derivatized. Typical examples of cationic protein hydrolysates and derivatives are the products known under the INCI designations and commercially available: Cocodimonium Hydroxypropyl Hydrolyzed Collagen, Cocodimopnium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed keratin, Hydroxypropyltrimonium Hydrolyzed Rice Bran Protein, Hydroxyproypltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

In an embodiment as contemplated herein the styling composition comprises at least two different protein hydrolysates and/or protein hydrolysate derivatives, preferably vegetable protein hydrolysates and/or protein hydrolysate derivatives, particularly preferably hydrolyzed wheat protein and hydrolyzed almond protein.

Preferably the at least one protein hydrolysate and/or a protein hydrolysate derivative with a proportion of from about 0.005 to about 1.0% by weight, preferably from about 0.007 to about 0.75% by weight, particularly preferably from about 0.01 to about 0.5% by weight, even more preferably from about 0.01 to about 0.35% by weight, based on the total proportion of the styling composition, in the styling composition.

In a particular embodiment as contemplated herein, in which the styling composition comprises at least two different protein hydrolysates and/or protein hydrolysate derivatives, a first protein hydrolysate or protein hydrolysate derivative is present in a proportion of from about 0.05 to about 0.7% by weight, preferably from about 0.1 to about 0.5% by weight, very particularly preferably from about 0.15 to about 0.4% by weight, of the styling composition. %, even more preferably from about 0.15 to about 0.35% by weight, and a second protein hydrolysate or protein hydrolysate derivative in a proportion of from about 0.004 to about 0.1% by weight, preferably from about 0.005 to about 0.09% by weight, very particularly preferably from about 0.007 to about 0.05% by weight, even more preferably from about 0.009 to about 0.03% by weight, based on the total proportion of the styling composition, in the styling composition.

Preferably, the styling composition contains further components chosen from surfactants, preservatives, humectants, agents for pH adjustment, care products, dyes, conditioning agents, perfume, and mixtures of these.

In a configuration of the present disclosure, the styling composition may additionally contain nonionic, anionic, cationic, and/or amphoteric or amphoteric surfactants. Examples of anionic surfactants are soaps, alkylbenzene sulfonates, alkane sulfonates, olefin sulfonates, alkyl ether sulfonates, glycerol ether sulfonates, α-methyl ester sulfonates, sulfo fatty acids, alkyl sulfates, fatty alcohol ether sulfates, glycerol ether sulfates, hydroxy mixed ether sulfates, monoglyceride (ether) sulfates, fatty acid amide (ether) sulfates, mono- and dialkyl sulfosuccinates, Mono- and dialkylsulfosuccinamates, sulfotriglyceride, amide soaps, ether carboxylic acids and their salts, fatty acid isethionates, fatty acid sarcosinates, fatty acid taurides, N-acyl amino acids, such as acyl lactylates, acyl tartrates, acyl glutamates and acyl spartates, alkyl oligoglucoside sulfates, protein fatty acid condensates (in particular wheat-based vegetable products) and alkyl (ether) phosphates. If the anionic surfactants contain polyglycol ether chains, these can have a conventional, but preferably a narrowed homologue distribution. Examples of non-ionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed-formal, optionally partially oxidized alk(en)yl oligoglycosides or glucoronic acid derivatives, fatty acid-N-alkyl glucamides, protein hydrolysates (in particular wheat-based vegetable products), polyol fatty acid esters, sugar esters, sorbitan esters, polysorbates and amine oxides. If the non-ionic surfactants contain polyglycol ether chains, these may have a conventional, but preferably a narrowed homologue distribution. Examples of cationic surfactants are quaternary ammonium compounds and esterquats, especially quaternized fatty acid trialkanolamine ester salts. Typical examples of amphoteric or zwitterionic surfactants are alkylbetaines, alkylamidobetaines, aminopropionates, aminoglycinate, imidazoliniumbetaines and sulfobetaines. The surfactants mentioned are exclusively known compounds. With regard to the structure and manufacture of these substances, reference is made to relevant reviews, for example J. Falbe (ed.), “Surfactants in Consumer Products”, Springer Verlag, Berlin, 1987, pp. 54-124 or J. Falbe (ed.), “Catalysts, Surfactants and Mineral Oil Additives”, Thieme Verlag, Stuttgart, 1978, pp. 123-217. The proportion of additional surfactants in the styling composition is preferably from about 0.05 to about 5.5% by weight, more preferably from about 0.1 to about 3.5% by weight, more preferably from about 0.2 to about 3.5% by weight, based on the total proportion of the styling composition.

Furthermore, the styling composition may contain agents for adjusting the pH value. Preferred agents are for example primary amino alcohols such as Aminomethyl Propanol (INCI), which is commercially available under the name AMP-ULTRA© PC, for example AMP-ULTRA© PC 2000. Other pH adjusting agents may be chosen from alkaline hydroxides, in particular sodium hydroxide and potassium hydroxide, organic acids such as lactic acid, citric acid, hydrochloric acid, sulfuric acid, phosphoric acid, adipic acid, malic acid, succinic acid, tartaric acid or malic acid, in particular tetrahydroxypropyl ethylenediamine known under the trade names Neutrol TE and triethylamine (TEA). The proportion of pH adjustment agents in the styling composition is preferably from about 0.01 to about 1.0% by weight, particularly preferably from about 0.05 to about 0.1% by weight, particularly preferably from about 0.05 to about 0.08% by weight, based on the total proportion of the styling composition.

The styling composition may additionally contain a conditioning agent such as a natural oil chosen from, for example, jojoba oil, almond oil, olive oil, grape seed oil, hemp oil, soybean oil, cotton seed oil, sunflower oil, palm oil, palm kernel oil, linseed oil, castor oil, corn oil, rapeseed oil, sesame oil, safflower oil, wheat germ oil, peach kernel oil, the liquid portions of coconut oil and mixtures thereof.

The proportion of conditioning agents in the styling composition is preferably from about 0.01 to about 5.0% by weight, more preferably from about 0.05 to about 3.0% by weight, more preferably from about 0.1 to about 1.0% by weight, based on the total proportion of the styling composition.

Furthermore, the styling composition may contain a preservative, in particular those listed in the Cosmetic Products Regulation in Annex V (Regulation (EC) No 122+3/2009 of the European Parliament and of the Council of 30 Nov. 2009 on cosmetic products), particularly preferably benzoic acid and benzoates and mixtures thereof.

The proportion of preservatives in the styling composition is preferably from about 0.05 to about 1.0% by weight, more preferably from about 0.075 to about 0.6% by weight, more preferably from about 0.1 to about 0.4% by weight, based on the total proportion of the styling composition.

The styling composition is preferably used for shaping keratinous fibers. It has been shown that the inventive combination of the components results in a styling composition that meets the currently increasing consumer demand for products with natural ingredients and is particularly suitable for the temporary reshaping of keratinous fibers, preferably human scalp hair, while keeping the hairstyle in the desired shape while retaining its lightness and appearing neither heavy nor stiff.

Some further preferred styling compositions can be found in the following tables (data in weight % based on the total weight of the styling composition unless otherwise stated).

| | | | | Formula | Formula |
|---|---|---|---|---|---|
| Components | Formula 1 | Formula 2 | Formula 3 | 4 | 5 |
| modified starch | 1.3-7.5 | 1.8-5.5 | 2.5-4.5 | 2.7-4.0 | 2.8-3.5 |
| natural plant | 0.2-6.5 | 0.7-5.0 | 1.3-3.5 | 1.5-3.0 | 1.9-2.5 |
| exudate | | | | | |
| and/or extract | | | | | |
| cationic guar | 0.03-1.3 | 0.06-0.9 | 0.09-0.85 | 0.15-0.7 | 0.25-0.04 |
| derivative | | | | | |
| optional | ad. 100 | ad. 100 | ad. 100 | ad. 100 | ad. 100 |
| components and | | | | | |
| distilled water | | | | | |

| Components | Formula 6 | Formula 7 | Formula 8 | Formula 9 | Formula 10 |
|---|---|---|---|---|---|
| Dextrin | 1.3-7.5 | 1.8-5.5 | 2.5-4.5 | 2.7-4.0 | 2.8-3.5 |
| Acacia | 0.2-6.5 | 0.7-5.0 | 1.3-3.5 | 1.5-3.0 | 1.9-2.5 |
| exudate | | | | | |
| and/or | | | | | |
| extract | | | | | |
| cationic guar | 0.03-1.3 | 0.06-0.9 | 0.09-0.85 | 0.15-0.7 | 0.25-0.04 |
| derivative | | | | | |
| optional | ad. 100 | ad. 100 | ad. 100 | ad. 100 | ad. 100 |
| components | | | | | |
| and distilled | | | | | |
| water | | | | | |

| | Formula | Formula | Formula | Formula | Formula |
|---|---|---|---|---|---|
| Components | 11 | 12 | 13 | 14 | 15 |
| Dextrin | 1.3-7.5 | 1.8-5.5 | 2.5-4.5 | 2.7-4.0 | 2.8-3.5 |
| Acacia exudate | 0.2-6.5 | 0.7-5.0 | 1.3-3.5 | 1.5-3.0 | 1.9-2.5 |
| and/or extract | | | | | |
| Guar | 0.03-1.3 | 0.06-0.9 | 0.09-0.85 | 0.15-0.7 | 0.25-0.04 |
| Hydroxypropyl- | | | | | |
| trimonium- | | | | | |
| chlorde | | | | | |
| optional | ad. 100 | ad. 100 | ad. 100 | ad. 100 | ad. 100 |
| components | | | | | |
| and distilled | | | | | |
| water | | | | | |

| | Formula | Formula | Formula | Formula | Formula |
|---|---|---|---|---|---|
| Components | 16 | 17 | 18 | 19 | 20 |
| Dextrin | 1.3-7.5 | 1.8-5.5 | 2.5-4.5 | 2.7-4.0 | 2.8-3.5 |
| Acacia exudate | 0.2-6.5 | 0.7-5.0 | 1.3-3.5 | 1.5-3.0 | 1.9-2.5 |
| and/or extract | | | | | |
| Guar | 0.03-1.3 | 0.06-0.9 | 0.09-0.85 | 0.15-0.7 | 0.25-0.04 |
| Hydroxypropyl- | | | | | |
| trimonium- | | | | | |
| chlorde | | | | | |
| optional | 0.1-15.0 | 0.5-13.0 | 0.8-12.0 | 1.0-11.0 | 1.5-10.0 |
| components | | | | | |
| distilled water | ad. 100 | ad. 100 | ad. 100 | ad. 100 | ad. 100 |

| Components | Formula 21 | Formula 22 | Formula 23 | Formula 24 | Formula 25 |
|---|---|---|---|---|---|
| modified starch | 1.3-7.5 | 1.8-5.5 | 2.5-4.5 | 2.7-4.0 | 2.8-3.5 |
| natural plant exudate | 0.2-6.5 | 0.7-5.0 | 1.3-3.5 | 1.5-3.0 | 1.9-2.5 |
| and/or extract | | | | | |
| cationic guar | 0.03-1.3 | 0.06-0.9 | 0.09-0.85 | 0.15-0.7 | 0.25-0.04 |
| derivative | | | | | |
| Protein hydrolysate | 0.006-0.85 | 0.008-0.7 | 0.009-0.6 | 0.01-0.4 | 0.01-0.35 |
| and/or protein | | | | | |
| hydrolysate derivative | | | | | |
| optional components | ad. 100 | ad. 100 | ad. 100 | ad. 100 | ad. 100 |
| and distilled water | | | | | |

| Components | Formula 26 | Formula 27 | Formula 28 | Formula 29 | Formula 30 |
|---|---|---|---|---|---|
| Dextrin | 1.3-7.5 | 1.8-5.5 | 2.5-4.5 | 2.7-4.0 | 2.8-3.5 |
| Acacia exudate and/or | 0.2-6.5 | 0.7-5.0 | 1.3-3.5 | 1.5-3.0 | 1.9-2.5 |
| extract | | | | | |
| cationic guar | 0.03-1.3 | 0.06-0.9 | 0.09-0.85 | 0.15-0.7 | 0.25-0.04 |
| derivative | | | | | |
| Protein hydrolysate | 0.006-0.85 | 0.008-0.7 | 0.009-0.6 | 0.01-0.4 | 0.01-0.35 |
| and/or protein | | | | | |
| hydrolysate derivative | | | | | |
| optional components | ad. 100 | ad. 100 | ad. 100 | ad. 100 | ad. 100 |
| and distilled water | | | | | |

| Components | Formula 31 | Formula 32 | Formula 33 | Formula 34 | Formula 35 |
|---|---|---|---|---|---|
| Dextrin | 1.3-7.5 | 1.8-5.5 | 2.5-4.5 | 2.7-4.0 | 2.8-3.5 |
| Acacia exudate and/or | 0.2-6.5 | 0.7-5.0 | 1.3-3.5 | 1.5-3.0 | 1.9-2.5 |
| extract | | | | | |
| Guar Hydroxypropyl- | 0.03-1.3 | 0.06-0.9 | 0.09-0.85 | 0.15-0.7 | 0.25-0.04 |
| trimoniumchlorde | | | | | |
| Protein hydrolysate | 0.006-0.85 | 0.008-0.7 | 0.009-0.6 | 0.01-0.4 | 0.01-0.35 |
| and/or protein | | | | | |
| hydrolysate derivative | | | | | |
| optional components | ad. 100 | ad. 100 | ad. 100 | ad. 100 | ad. 100 |
| and distilled water | | | | | |

| Components | Formula 36 | Formula 37 | Formula 38 | Formula 39 | Formula 40 |
|---|---|---|---|---|---|
| Dextrin | 1.3-7.5 | 1.8-5.5 | 2.5-4.5 | 2.7-4.0 | 2.8-3.5 |
| Acacia exudate and/or | 0.2-6.5 | 0.7-5.0 | 1.3-3.5 | 1.5-3.0 | 1.9-2.5 |
| extract | | | | | |
| Guar Hydroxypropyl- | 0.03-1.3 | 0.06-0.9 | 0.09-0.85 | 0.15-0.7 | 0.25-0.04 |
| trimoniumchlorde | | | | | |
| Protein hydrolysate | 0.006-0.85 | 0.008-0.7 | 0.009-0.6 | 0.01-0.4 | 0.01-0.35 |
| and/or protein | | | | | |
| hydrolysate derivative | | | | | |
| optional components | 0.1-15.0 | 0.5-13.0 | 0.8-12.0 | 1.0-11.0 | 1.5-10.0 |
| distilled water | ad. 100 | ad. 100 | ad. 100 | ad. 100 | ad. 100 |

| Components | Formula 41 | Formula 42 | Formula 43 | Formula 44 | Formula 45 |
|---|---|---|---|---|---|
| Organic birch juice | 0.5-9.0 | 1.0-7.5 | 1.5-6.0 | 2.5-5.5 | 5.1 |
| Agenamalt 20.233 | 0.5-7.0 | 1.0-5.5 | 1.5-4.5 | 2.0-4.0 | 3.0 |
| Instant Gum AA | 0.1-6.0 | 0.7-4.5 | 0.9-3.5 | 1.0-3.0 | 2.0 |
| Organic | | | | | |
| Sodium benzoate | 0.05-1.5 | 0.1-1.0 | 0.1-0.9 | 0.2-0.5 | 0.3 |
| Glycerin 4811 | 0.5-5.5 | 0.9-4.0 | 1.0-2.5 | 1.3-2.0 | 1.5 |
| Gluadin WLM Benz | 0.5-5.5 | 0.9-4.0 | 1.0-2.5 | 1.3-2.0 | 1.5 |
| Gluadin Almond Benz | 0.005-1.0 | 0.009-0.5 | 0.01-0.3 | 0.03-0.09 | 0.05 |
| JAGUAR EXCEL | 0.05-1.8 | 0.1-1.3 | 0.2-0.9 | 0.3-0.6 | 0.4 |
| (sustainable) | | | | | |
| Perfume Top Biobell | 0.03-1.4 | 0.08-0.9 | 0.1-0.7 | 0.15-0.4 | 0.2 |
| AF W 19612 | | | | | |
| Lactic acid 80% Prod | 0.01-1.1 | 0.05-0.7 | 0.07-0.5 | 0.08-0.2 | 0.1 |
| 132308 (young polish | | | | | |
| pottery) | | | | | |
| distilled water, without | ad. 100 | ad. 100 | ad. 100 | ad. 100 | ad. 100 |
| H₂O₂ | | | | | |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims. It is contemplated that the terminology “about” when referencing a range refers to both “about” the lower value of the range and/to “about” the higher value of the range.

What is claimed is:

1. Styling composition for temporarily deforming keratinous fibers comprising
a) at least one modified starch,
b) at least one natural plant exudate and/or extract, and
c) at least one guar derivative.

2. Styling composition according to claim 1, wherein the at least one natural plant exudate and/or extract is an acacia exudate and/or extract.

3. Styling composition according to claim 1, wherein the at least one guar derivative is a cationic guar derivative.

4. Styling composition according to claim 1, wherein the at least one modified starch is included in the styling composition in a proportion of from about 1.0 to about 8.0% by weight.

5. Styling composition according to claim 1, wherein the at least one natural plant exudate and/or extract is included in the styling composition in a proportion of from about 0.1 to about 7.0% by weight.

6. Styling composition according to claim 1, wherein the at least one guar derivative is included in the styling composition at a proportion of from about 0.01 to about 1.5% by weight.

7. Styling composition according to claim 1, wherein the composition includes at least one protein hydrolysate and/or one protein hydrolysate derivative.

8. Styling composition according to claim 1, wherein the at least one protein hydrolysate and/or protein hydrolysate derivative is included in the styling composition at a proportion of from about 0.005 to about 1.0% by weight.

9. Styling composition according to claim 1, wherein the composition includes further components chosen from surfactants, preservatives, humectants, pH adjusting agents, care agents, dyes, conditioning agents, perfume, and mixtures thereof.

10. Styling composition according to claim 1, wherein the at least one guar derivative is hydroxypropyltrimonium chloride.

11. Styling composition according to claim 1 wherein the at least one modified starch is included in the styling composition in a proportion of from about 2.0 to about 4.0% by weight based on the total proportion of the styling composition.

12. Styling composition according to claim 2 wherein the at least one modified starch is included in the styling composition in a proportion of from about 2.0 to about 4.0% by weight based on the total proportion of the styling composition.

13. Styling composition according to claim 1 wherein the at least one natural plant exudate and/or extract is included in the styling composition in a proportion of from about 1.5 to about 3.0% by weight based on the total proportion of the styling composition.

14. Styling composition according to claim 2 wherein the at least one natural plant exudate and/or extract is included in the styling composition in a proportion of from about 1.5 to about 3.0% by weight based on the total proportion of the styling composition.

15. Styling composition according to claim 1, wherein the at least one modified starch is included in the styling composition in a proportion of from about 2.0 to about 5.0% by weight.

16. Styling composition according to claim 1, wherein the at least one natural plant exudate and/or extract is included in the styling composition in a proportion of from about 1.0 to about 4.0% by weight.

17. Styling composition according to claim 1, wherein the at least one guar derivative is included in the styling composition at a proportion of from about 0.1 to about 0.8% by weight.

18. Styling composition for temporarily deforming keratinous fibers comprising
a dextrin present in an amount of from about 2.0 to 4.0% by weight, based on the total proportion of the styling composition,
acacia exudate and/or extract present in an amount of from about 1.5 to 3.0% by weight, based on the total proportion of the styling composition, and
guar hydroxypropyltrimonium chloride present in an amount of from about 0.2 to 0.5% by weight, based on the total proportion of the styling composition,
at least one protein hydrolysate and/or one protein hydrolysate derivative present in an amount of from about 0.01 to 0.35% by weight, based on the total proportion of the styling composition, and
one or more components chosen from surfactants, preservatives, humectants, pH adjusting agents, care agents, dyes, conditioning agents, perfume, and mixtures thereof.
